(19) United States
(12) Patent Application Publication
(10) Pub. No.: US 2014/0329716 A1
(43) Pub. Date: Nov. 6, 2014

(54) DEVICES HAVING A CALIBRATION CONTROL REGION, SYSTEMS AND METHODS FOR IMMUNOHISTOCHEMICAL ANALYSES

(71) Applicants: Brad A. Kairdolf, Lilburn, GA (US); Shuming Nie, Atlanta, GA (US)

(72) Inventors: Brad A. Kairdolf, Lilburn, GA (US); Shuming Nie, Atlanta, GA (US)

(73) Assignee: EMORY UNIVERSITY, Atlanta, GA (US)

(21) Appl. No.: 14/241,475

(22) PCT Filed: Aug. 28, 2012
(86) PCT No.: PCT/US2012/052604

§ 371 Date: Jul. 29, 2014

Related U.S. Application Data
(60) Provisional application No. 61/530,484, filed on Sep. 2, 2011.

(51) Int. Cl. G01N 33/574

(52) U.S. Cl. CPC G01N 33/57407
USPC 506/10; 435/287.2; 506/14; 435/7.1

(57) ABSTRACT

The disclosure relates to devices, systems and methods that address the variability in immunohistochemistry (IHC) tests that can lead to inaccurate diagnosis or misdiagnosis while maintaining important structural information within the specimen. The devices may include a control region having a control unit including a plurality of substantially homogenous samples and a biological sample region.

### CROSS REFERENCE TO RELATED APPLICATION

This application claims priority to U.S. Provisional Application No. 61/530,484 filed Sep. 2, 2011, hereby incorporated by reference in its entirety.

### ACKNOWLEDGEMENT

This invention was made with government support under Grant No. CA119338, awarded by the National Institutes of Health. The government has certain rights in the invention.

### BACKGROUND

Accurate and early diagnosis is a primary predictor of overall patient survival, dramatically reducing mortality rates in many diseases Immunohistochemical (IHC) stains have been an integral part of the diagnosis process.

However, while considerable progress has been made in the improvement of some diagnostic technologies, inaccuracies and errors in clinical diagnostics remain a significant challenge to improving patient care. This is particularly true in pathology, where analysis and diagnosis is subjective and requires the experience of a trained physician. In fact, erroneous cytopathology reports spurred the passage of the Clinical Laboratory Improvement Amendments (CLIA), implementing federal regulatory standards for all labs performing diagnostic testing in the US.

Quality control is mandated by those amendments. Many immunohistochemical (IHC) techniques incorporate such controls. See, for example, U.S. Pat. No. 6,281,004; U.S. Pat. No. 7,560,282; U.S. Pat. No. 7,598,036; and International Publication Number WO 2012/078138.

Despite these efforts, the qualitative nature of techniques, such as immunohistochemistry (IHC) and the variability in staining and interpretation across diagnostic labs, has contributed substantially to errors in clinical diagnoses, which can be 11% or higher in some cases. Nearly half of these errors result in harm to the patient, including delayed or unnecessary procedures, or missed diagnoses. These errors also result in increased health care costs from additional diagnostic tests and inappropriate administration of expensive therapies (e.g., up to 18% of breast cancer patients may receive Her2 targeted therapy unnecessarily at a cost of $100 k).

### SUMMARY

Thus, there is a need for technology that can accurately quantify profile cell samples from patients.

The disclosure relates to devices having a calibration control region, systems and methods for assays that measure analytes in cells or tissue sections, specifically immunohistochemical analyses of biological or cytological samples. The disclosure relates to real-time quality control.

The disclosure may relate to a device configured for immunohistochemical tests of a biological sample using at least one reporter specific to at least one biomarker. The device may include a substrate, the substrate including a control region and a biological sample region, the control region including a control unit, the control unit including a plurality of control samples of pseudo-tissue, each control sample including at least one of a first material and a second material, the first material including one or more homogenates that are negative for the at least one biomarker, and the second material including one or more homogenates that are positive for the at least one biomarker. In some embodiments, each sample may be of different concentrations of the first and second materials. In other embodiments, the sample may be disposed on the control unit in order of highest negative concentration to lowest positive concentration.

In some embodiments, the control unit may further include at least one replicate sample for each sample. The control samples and replicate samples may be disposed in a microarray. In some embodiments, the samples may be disposed on the control unit in order of highest negative concentration to lowest positive concentration in a first direction. In further embodiments, each replicate sample may be disposed on the control unit in a second direction adjacent to respective control sample. In some embodiments, each sample of the control unit may be substantially homogenous. In further embodiments, each sample of the control unit may be configured to have substantially uniform intensity so that each pixel within the sample is substantially the same. In other embodiments, the image of each sample of the control unit may have a substantially uniform appearance. In some embodiments, each control sample may be preserved according to the preparation process used in clinical specimens.

In some embodiments, the control unit may include at least three different control samples of differing concentrations. The first control sample may include all of a first material. The second control sample may include one-half of the first material and one-half of the second material. The second control sample may include all of the second material. In some embodiments, the control samples may be configured to test one biomarker. In some embodiments, the control samples may be configured to test two different biomarkers.

In some embodiments, the disclosure may relate to a kit for immunohistochemical tests of a biological sample using at least one reporter. The kit may include a plurality of control devices; each device including: a substrate, the substrate including a control region and a biological sample region, the control region including a control unit, the control unit including a plurality of control samples of pseudo-tissue, each control sample including at least one of a first material and a second material, the first material including one or more homogenates that are negative for the at least one biomarker to be tested, and the second material including one or more homogenates that are positive for the at least one biomarker to be tested. In some embodiments, the kit may include the at least one reporter. In further embodiments, the at least one reporter may be quantum dots.

In some embodiments, the disclosure may relate to a method for determining a concentration of at least one biomarker in a biological sample on a control device. In further embodiments, the method may be computer-implemented. In other embodiments, the disclosure may relate to a computer-readable medium stored with instructions to cause a computer to execute the method for determining a concentration of at least one biomarker in a biological sample on a control device.

In some embodiments, the method may include receiving an image of the control device prepared with at least one reporter specific to each at least one biomarker, the image including an image of the biological sample and of a control unit, the control unit including a plurality of control samples of a pseudo-tissue of different concentrations of the at least one biomarker, and at least one replicate sample for each control sample; analyzing the image of the control unit, the analyzing the image of the control unit include determining the intensities of each control and replicate sample and averaging the intensities of control and replicate samples for each concentration; correlating the averaged intensities to each concentration; analyzing the image of the biological sample to determine intensity of at least one region; comparing the intensity of the at least one region to the correlated intensities and concentrations of the control unit; and determining the concentration of the biomarker in the at least one region; and reporting the concentration of the biomarker.

In some embodiments, each sample of the control unit is configured to have substantially uniform intensity throughout the sample. In some embodiments, more than one biomarker may be analyzed simultaneously. In further embodiments, the at least one reporter is a fluorescent stain specific to each biomarker; and the correlating includes simultaneously correlating the averaged intensities for each biomarker to each concentration.

In some embodiments, each control sample has a homogenous appearance. The reporter may be quantum dots. In further embodiments, the reporting may include generating a concentration profile of the biological sample.

### DESCRIPTION OF FIGURES

The disclosure can be better understood with the reference to the following drawings and description. The components in the figures are not necessarily to scale, emphasis being placed upon illustrating the principles of the disclosure.

FIG. 1A shows an example of Her-2 staining in a breast ductal carcinoma. Intense staining (left) is consistent with results from FISH. Insufficient marker staining (right) can result from errors or variability in the protocol and can lead to misdiagnosis (adapted from nordiqc.org).

FIG. 1B shows an example of fine needle aspiration (FNA, left), which is a routine and safe biopsy procedure. Minute amounts of sample are obtained however (right), making quantitative molecular profiling a challenge.

FIG. 2 shows an example of a control device for immunohistochemical analyses according to embodiments.

FIG. 3A shows an example of a device having control unit (e.g., PTMA) on the left. The samples (including replicates) are designed with controlled marker concentrations (right) for simultaneous staining calibration of multiple markers (red & green fluorescence). FIG. 3B shows two curves: the correlation of staining intensity (left) to known marker concentrations, and the standard curves (right).

FIG. 4A shows IHC staining to visualize biomarker expression in cell/tissue based standards. Significant variability in staining intensity (>25% when averaged) is revealed due to biomarker localization and the overall heterogeneity of the control. FIG. 4B shows fluorescence staining of ER in three separately prepared PTMA control spots. Intensity plots show uniform staining across the entire spot and highly repeatable staining intensity (varying <1%), important for use as a quantitative staining control.

FIG. 5 shows a method of determining concentrations of a sample using the control device according to embodiments.

FIG. 6 shows a method of analyzing the sample on the device according to embodiments.

FIG. 7 shows an example of a schematic workflow using the devices and computer-implemented methods according to the embodiments.

FIG. 8 shows an example of analysis of a Hodgkin's lymphoma specimen using fluorescent quantum dots on a control unit according to the embodiments. Multicolor imaging in a Hodgkin's Lymphoma specimen using fluorescent quantum dots is shown on the left. Image analysis allows simultaneous detection of multiple fluorescence signals in a single specimen slide (middle) for multimarker detection (right, CD45/CD30/Pax5) and molecular profiling of single cells.

FIGS. 9A and 9B shows an example of studies for single and double marker analysis, respectively. FIG. 9A shows a single marker analysis of estrogen receptor (ER) in a PTMA control prepared from negative (N) and positive (P) expression homogenates. Spot size and staining intensity are uniform and repeatable for all concentrations in the PTMA, showing high correlation to marker concentration (bottom). FIG. 9B shows a magnified image of 4 PTMA spots showing multimarker analysis of ER and Her2 using red and green QDs, and the generation of standard curves (bottom) for quantitative profiling in the sample.

FIG. 10 shows a schematic showing the steps of a fabrication process of the control unit.

FIG. 11 shows an example of a system according to embodiments.

### DESCRIPTION OF EMBODIMENTS

The disclosure relates to devices, systems and methods that address the variability in immunohistochemistry (IHC) tests that can lead to inaccurate diagnosis or misdiagnosis while maintaining important structural information within the specimen. The variability may be introduced by many sources, such as human error, sample preparation, subjective analyses, or instrumentation.

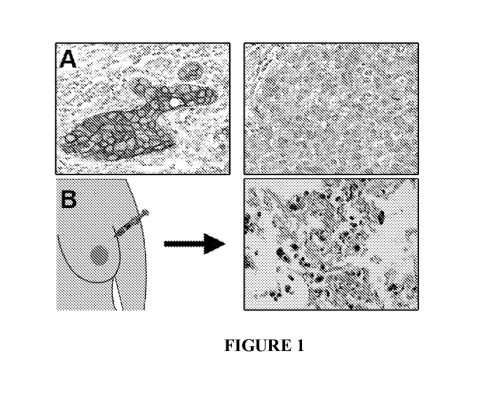

Marker stain intensity is one example of variability. FIG. 1A shows a comparison of two images of HER-2 staining in a breast ductal carcinoma. The image on the left shows intense staining while the image on the right shows insufficient marker staining. However, this assay utilizes separate control slides with cancer cell lines for assay validation and is only semi-quantitative, relying on a subjective analysis by the pathologist to estimate marker expression. Even using this control technique, suboptimal staining occurs in about 25% of samples with over 11% of cases presenting substantial inaccuracies. While some companies have incorporated hardware and software tools to measure samples in an objective way, they still employ the use of heterogeneous whole cell or tissue standards, where it is impossible to tune biomarker content for robust standard curves, accurately normalize variability, and correlate staining intensity to marker expression in patient samples.

Another example of variability includes subjective analyses of controls for IHC tests. One common control technique is actual tissue microarrays. However, actual tissue microarrays are also poor controls because they are costly, can only be obtained from human patients, and must be pre-analyzed by a pathologist (still qualitative).

Also, there may be too little amount of material obtained from the patient to have for the analyses and controls. Clinical IHC assays analyze just a single marker, making their utility in high throughput molecular profiling limited. One example is needle biopsies (FNAs), a pain-free and inexpensive procedure that typically contains too little material for a multimarker profile using existing products. For example, FIG. 1B shows an example of fine needle aspiration for breast tissue. Examples of minute samples of breast tissue are shown on the right obtained from the breast shown on the left. This functionality is increasingly important with the advent of personalized medicine, where treatment is tailored to the patient based on their unique molecular profile. Accurate molecular profiling may successfully predict whether a patient will respond positively to some targeted drugs, such as cetuximab (EGFR) and trastuzumab (Her2).

The disclosure relates to devices, systems, and methods allow for real-time quality control and multiplexed biomarker quantification in structurally intact specimens. The disclosure relates to devices that incorporate a region for a control unit and a region for the cytology or biological sample (may also be referred to as “specimen”) to be analyzed. The control unit may be configured to analyze any number and type of biomarkers. In some embodiments, the control array may be configured to analyze at least one biomarker. In other embodiments, the control array may be configured to analyze two biomarkers. In further embodiments, the control array may be configured to analyze more than two biomarkers.

Although the examples relate to estrogen receptors (ER) and Her2, it is understood that device may be configured to analyze any number and type of biomarker.

### Control Device

According to some embodiments, the disclosure relates to a control device having a control unit including control samples with known biomarker concentrations or levels. The control unit may be placed on the device along with the patient sample to be tested. This enables calibration of an immunohistochemical reporter (e.g., staining, quantum dots, etc.) on a sample by sample basis.

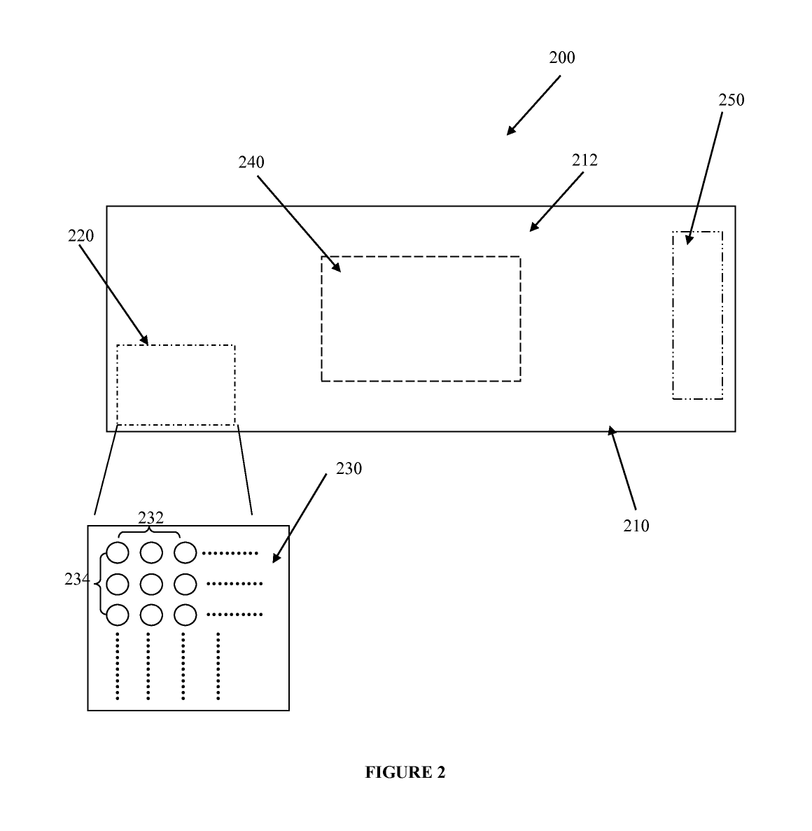

FIG. 2 shows a device 200 according to embodiments. The device 200 may include a substrate 210. The substrate may be of rectangular shape. In other embodiments, the substrate may be of any shape. The substrate 210 may include a first surface 212 and an opposing second surface (not shown). In some embodiments, the substrate 210 may be made of a transparent surface, such as glass or have a glass surface. In further embodiments, the substrate 110 may be a microscopic slide.

The device 200 may further include a control region 220 and a tissue specimen region 230 on the first surface 212 as shown in FIG. 2. In some embodiments, the control region 220 may be disposed close to a side of the substrate. For example, as shown in FIG. 2, the control region 220 may be disposed close to the left side of the substrate 210. In other embodiments, the control region 220 may be disposed anywhere on the substrate 210. In some embodiments, the control region 220 may be disposed next to the biological or cytological sample region 240. In some embodiments, the biological or cytological sample (specimen) region 240 may be disposed in about the center of the substrate 210, for example, as shown in FIG. 2. In other embodiments, the biological sample region 240 may be disposed anywhere on the slide. The biological sample region 240 may be configured to hold any biological sample to be tested. The biological sample may include but is not limited to a cell, a group of cells, a tissue section, an array of tissue sections (e.g., a micro-tissue array), and any combination of one or more of any of these examples.

In further embodiments, the device 200 may further include a biological sample information section 250. The section 250 may include an area to include any information associated with the sample, the analysis of the sample, and the source of the sample. The information may include but is not limited to type of reporter used, surgical accession number, patient name or record number, a bar code, date, pathologist to perform the analysis. The information may be added to the area via a label or any known means.

The control section 220 may include a (calibration) control unit 230. In some embodiments, the control unit 230 may include a plurality of control samples. The plurality of control samples may be disposed in an array. The plurality of control samples may be disposed at separate locations in an ordered manner. The ordered manner may be dispositions at columns and rows. In some embodiments, each of the samples may be of uniform or substantially uniform size and shape. In other embodiments, the samples may be of different sizes and shapes.

In some embodiments, each of the plurality of samples 232 may be composed of a tissue material(s). In some embodiments, the tissue material may be pseudo-tissue. Pseudo-tissue (PT) may be considered any tissue-like cell aggregate that structurally and/or functionally resembles true tissue. The pseudo-tissue according to embodiments may have the properties of tissue without the morphology.

In some embodiments, each of the plurality of samples may be in a form of spot. In some embodiments, each sample of the control unit may be substantially homogenous with no spatial variability. Each sample may be designed so as to have substantially the same or uniform staining intensity throughout the sample (i.e., over the entire area of the sample) so as to be considered homogenous. The samples may be designed so that each pixel of the sample is substantially the same thereby having uniform staining intensity throughout the sample.

In some embodiments, the pseudo-tissue may be composed of a combination of homogenized cellular materials and a biological solidifying agent to produce substantially homogeneous samples with no spatial variability. In some embodiments, the cellular materials may be made from cell lines that exhibit very high expression and very low expression levels for the biomarker(s) to be analyzed. The cell lines may be any known cell lines. According to some embodiments, the cell lines may be grown in large quantities and used to prepare cellular materials containing high and low levels of protein expression, which may be verified using a known quantitative method. The materials may be combined in an appropriate combination (described below). In some embodiments, the solidifying agent may be any known agent. In some embodiments, the agent may include at least one of agarose, gelatin and collagen.

The combination of homogenized cellular materials may include one or both of the following cellular materials: (1) a first material including one or more homogenates that are negative for the marker(s) to be analyzed (e.g., a negative control); and (2) a second material including one or more homogenates that are positive for the marker(s) to be analyzed (e.g., positive control). The combination of homogenized cellular materials may depend on the type and number of biomarkers to be analyzed. For example, if one biomarker is to be analyzed, the first material may include one or more homogenates that are negative for that biomarker, and a second material may include one or more homogenates that are positive for that biomarker. If two or more biomarkers are to be analyzed, the first material would include one or more homogenates that are negative for all biomarkers, and a second material may include one or more homogenates that are positive for all biomarkers.

In some embodiments, the control samples of pseudo-tissue may be disposed in a micro-array (and may be later referred to as a pseudo tissue microarray (PTMA)). Some of the control samples may be of different pseudo tissue. Some of the control samples may differ in combinations of homogenates. In some embodiments, the combinations of homogenates may differ in biomarker(s) concentrations. The biomarker(s) concentrations may differ in varying ratios or concentrations. Although the biomarker(s) concentrations are described in varying ratios or percentages, it would be understood that the different combinations of the homogenates may be described in their concentrations. It would be further understood that the concentrations and ratios are not limited to those discussed below with respect to the figures. The control unit may include any number of differing concentrations and ratios of homogenates.

According to embodiments, each pseudo-tissue may be preserved to match the preparation process used in clinical specimens. In some embodiments, the pseudo-tissue may be preserved using formalin-fixation and paraffin embedding (FFPE). Cores taken from PTs (with known expression levels covering a wide range) may then be arranged to create a control unit. In some embodiments, the control unit may be a microarray of control samples (or spots). The block is then sectioned and mounted onto substrates, such as glass slides.

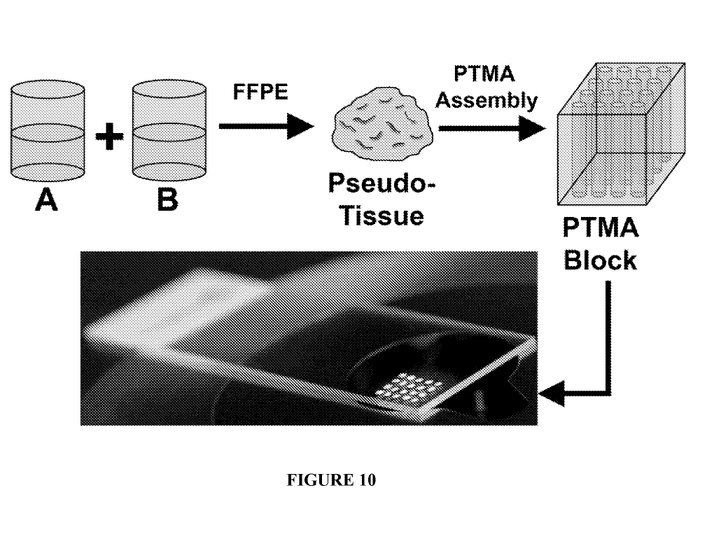

FIG. 10 shows a schematic of some of the steps of a fabrication process for the control unit according to some embodiments. Cellular homogenates (lysates) with low (A) and high (B) biomarker concentration may be combined in precise ratios to tune the biomarker concentration of the final control samples. Then, solidifying agents (ex. collagen) may be added to generate a pseudo-tissue, which is preserved using standard methods (ex. FFPE). Cores taken from control samples with different biomarker concentrations are then combined in a microarray format for sectioning and mounting onto a substrate, such as a slide.

In some embodiments, the control unit may include at least three control samples of different concentrations of the homogenates: a sample includes 100% of the first material (a homogenate that is negative for the marker(s) to be analyzed); a sample that includes 50% of the first material and 50% of the second material (a homogenate that are positive for the marker(s) to be analyzed) or a 2:2 ratio; and a sample that includes 100% of the second material. In other embodiments, the control unit may include more or less samples of different concentrations. For example, as shown in the figures, the control unit may include four or five different concentrations.

The different samples 232 may be disposed adjacent to each other in order of concentration of biomarker(s). In some embodiments, the different samples may be disposed adjacent to each other in order of increasing concentration. In some embodiments, the different samples may be disposed in different positions along a single row, as shown in the figures. In other embodiments, the different samples may be disposed in different positions along a single column.

In further embodiments, the control unit may further include at least one replicate (sample) 234 of each control sample that is disposed adjacent to the respective sample. The replicate(s) may be used to generate statistical information when performing immunohistochemical analyses using the device. In some embodiments, the control unit may include at least three replicates for each control sample. In other embodiments, the control unit may include more or less replicates.

In some embodiments, the at least one replicate(s) may be disposed in the same column respective sample but in a different row, as shown in the figures. In other embodiments, the replicate sample(s) may be disposed in different positions along a single row if the different samples are disposed in a single column.

According to embodiments, each device for specimen analysis may include a control unit, rather than using separate control slides. This allows for real-time quality control and multiplexed biomarker quantification in structurally intact specimens. The control unit may include a plurality of substantially uniform and homogeneous samples and tunable biomarker content.

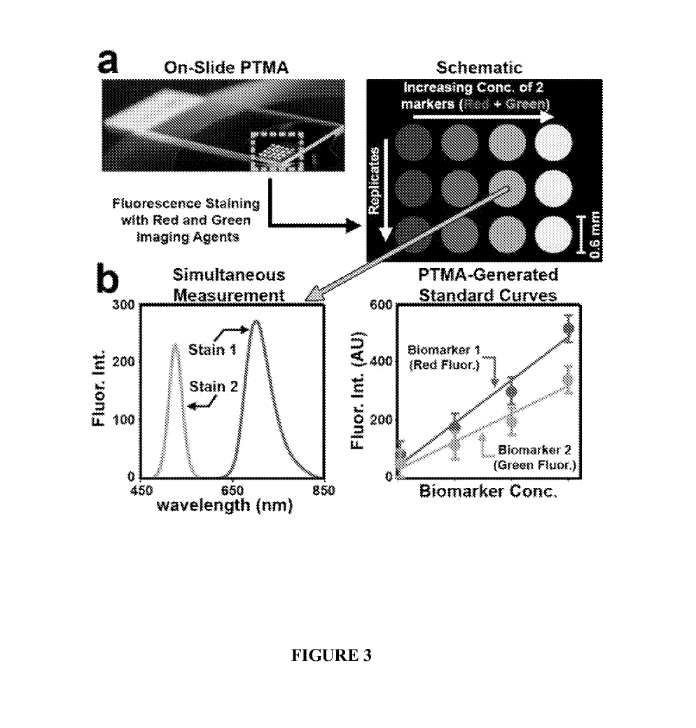

FIG. 3A shows an example of a device including a control unit in a form of a PTMA. The control unit may include samples including replicates with controlled marker concentrations for two biomarkers with increasing concentration moving from left to right. The increasing concentration is shown in the increasing intensity of color from left to right. This allows for simultaneous staining calibration of multiple markers.

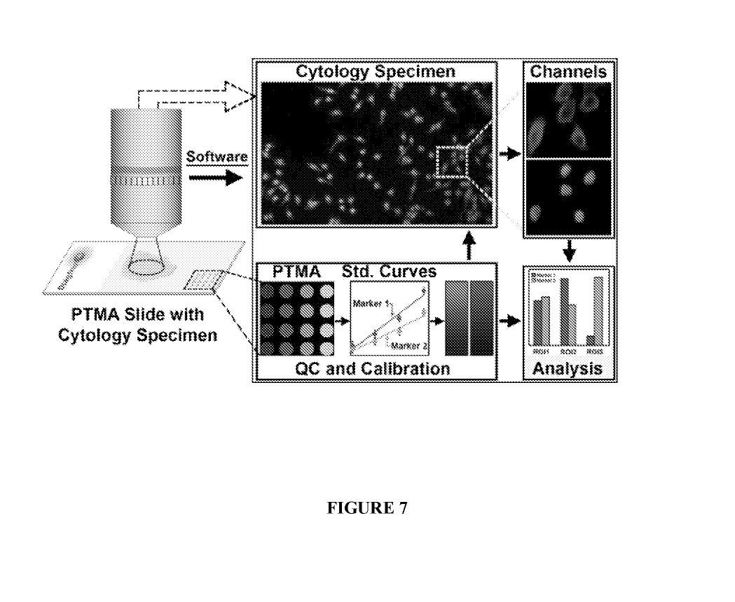

This tunable feature also enables the flexible design of precise standard curves for accurate and repeatable quantification of multiple markers in the specimen as shown in FIG. 3B. Thus, the multi-imaging capability of the device allows for correlation of staining intensity (shown in the left) to known marker concentrations, and the generation of robust standard curves (shown in the right), as shown in FIG. 7.

By combining two different homogenates in varying ratios (one with high marker concentrations and one that is negative for the markers), the biomarker concentrations in each pseudo-tissue can be precisely tuned (analogous to serial dilution of known standards in solution-based analytical assays). By combining these materials in varying ratios as disclosed, the control units may be used for accurate quantification of all biomarkers of interest.

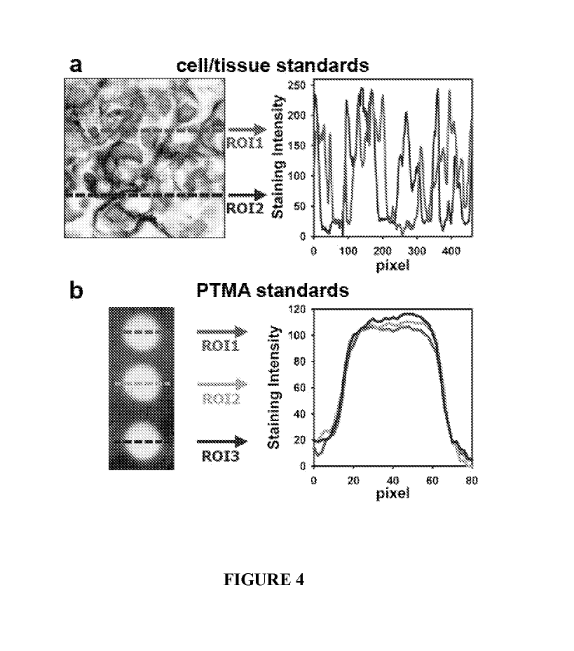

With existing products based on whole cells/tissue like those discussed above, it is impossible to alter biomarker content, making accurate and reproducible standard curve generation a major challenge. Separate control materials must be obtained or developed for each standard concentration (of each marker) desired, with the resulting data points often poorly spaced and covering the intensity range unevenly. This can lead to inaccuracies in the regression analysis and invalid standard curves. Marker staining can also extremely variable in these products due to the heterogeneity of protein expression (as shown in FIG. 4A). The high variability prevents the use of these products as accurate staining controls for quantitative molecular profiling of clinical specimens.

In contrast, the disclosed control unit may be substantially homogeneous or uniform and therefore have substantially the same or uniform intensity throughout each sample (e.g., over the entire area), as shown in FIG. 4B. The control unit may vary by <1%. The pseudo-tissue also serves as an excellent mimic for a number of inherent variables affecting sample analysis, including steric hindrance, nonspecific binding and autofluorescence. These contribute to the staining intensity observed in clinical samples and must be adequately controlled for to yield accurate results. Other control technologies (such as small peptides) cannot simulate these factors and are poor analogs for patient specimens.

Another advantage of the devices according to the embodiments is the multiplexing capabilities. Developing controls that can act as standards for multiple markers simultaneously can be a monumental technical challenge when using whole cell/tissue-based controls. However, to manufacture the devices according to synthetic process described, only two cellular materials are needed:
(1) one or more homogenates that are negative for all markers to be tested; and
(2) one or more homogenates that are positive for all markers to be tested.
   By combining these materials in varying ratios as described above, control units may be produced that can be used for accurate quantification of all biomarkers of interest.

### Systems & Methods of Quantifying Biomarker Concentration

According to embodiments, the control unit may be used as a concentration reference or control standard for expression analysis, e.g., quantitative of an analyte or marker concentration in a sample. The control unit may be used with a system configured to perform immunohistochemical analyses.

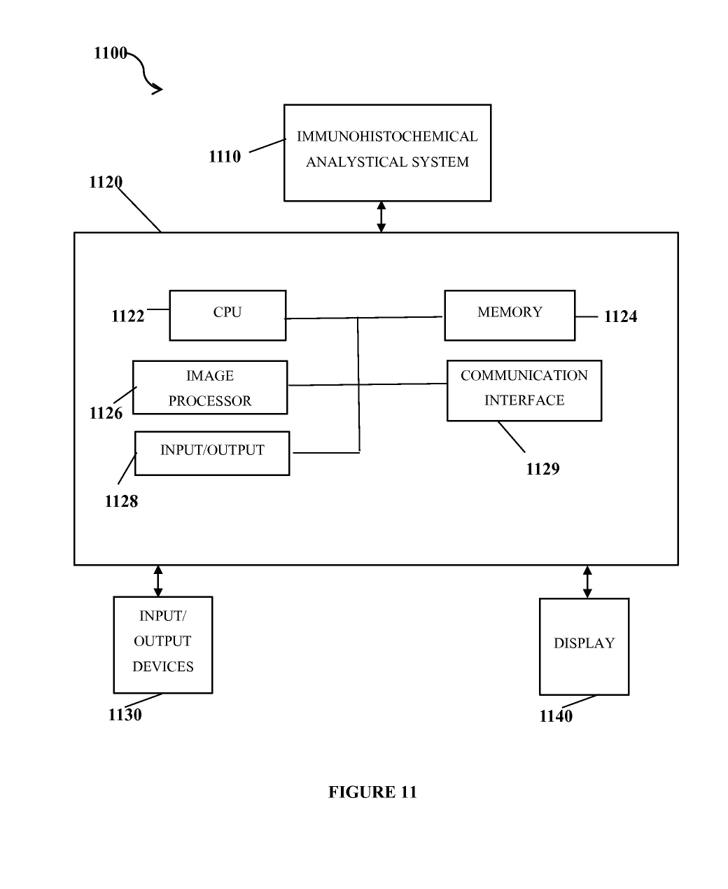

In some embodiments, the system may include an immunohistochemical analytical system and a computer system. An example of a system 1100 that includes an immunohistochemical analytical system 1110 and a computer system 1120 can be found in FIG. 11. The immunohistochemical analytical system 1110 may include instrumentation specific to the biomarker(s) to be analyzed and the reporters used. In some embodiments, the system 1110 may include a microscope system that includes a lighting device configured to illuminate the control device, optics configured to produce a magnified image of the illuminated target sample, a detector, such as a digital camera, configured to capture and generate a digital image of the magnified image. The system 1110 may also include fluorescence imaging or spectral hardware.

The computer system 1120 may be part of the immunohistochemical analytical system or may be in communication with the system via a network. The computer system 1120 may be configured to perform the analyses of the control unit and biological sample provided on the control device. The computer system may further be used to control the operation of the system or a separate system may be included.

The computer system 1120 may include a number of modules that communicate with each other through electrical and/or data connections (not shown). Data connections may be direct wired links or may be fiber optic connections or wireless communications links or the like. The computer system may also be connected to permanent or back-up memory storage, a network, or may communicate with a separate system control through a link (not shown). The modules may include a CPU 1122, a memory 1124, an image processor 1126, an input/output interface 1128 (e.g., printer interface), and a communication interface 1129. In addition, various other peripheral devices may be connected to the computer system such as an additional data storage device, a display device 1140, as well as any I/O devices 1130, such as a printer device. The computer system may also be connected to other computer systems as well as a network.

The CPU 1122 may be one or more of any known central processing unit, including but not limited to a processor, or a microprocessor. The CPU may be coupled directly or indirectly to memory elements. The memory may include random access memory (RAM), read only memory (ROM), disk drive, tape drive, etc., or a combinations thereof). The memory may also include a frame buffer for storing image data arrays.

The processes described below may be implemented as a routine that is stored in memory and executed by the CPU. As such, the computer system may be a general purpose computer system that becomes a specific purpose computer system when executing the routine of the disclosure. The computer system may also include an operating system and micro instruction code. The various processes and functions described herein may either be part of the micro instruction code or part of the application program or routine (or combination thereof) that is executed via the operating system.

The input/output 1128 may be configured for receiving information from one or more input devices 1130 (e.g., a keyboard, a mouse, and the like) and/or conveying information to one or more output devices 1140 (e.g., a printer, a CD writer, a DVD writer, portable flash memory, etc.).

The communication interface 1129 may be configured to conduct receiving and transmitting of data between other modules on the system and/or network. The communication interface 1129 may be a wired and/or wireless interface, a switched circuit wireless interface, a network of data processing devices, such as LAN, WAN, the internet, or combination thereof. The communication interface 1129 may be configured to execute various communication protocols, such as Bluetooth, wireless, and Ethernet, in order to establish and maintain communication with at least another module on the medical facility network.

It is to be understood that the embodiments of the disclosure be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the method steps depicted in the accompanying figures may be implemented in software, the actual connections between the systems components (or the process steps) may differ depending upon the manner in which the disclosure is programmed Given the teachings of the disclosure provided herein, one of ordinary skill in the related art will be able to contemplate these and similar implementations or configurations of the disclosure.

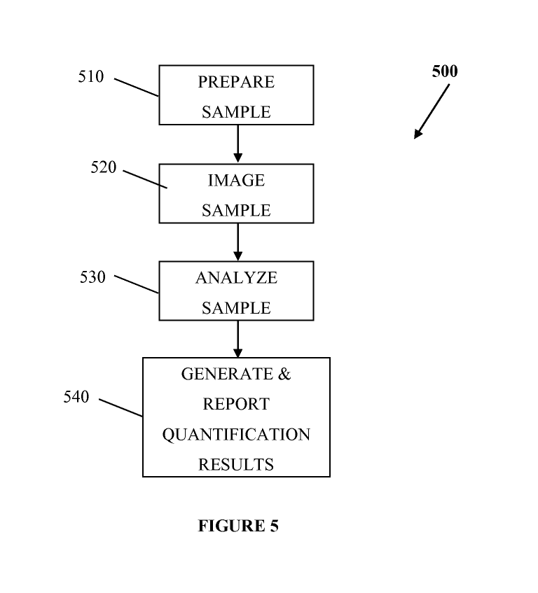

FIG. 5 shows a method of determining a concentration of a marker by generating a quantification report in a sample according to some embodiments. It will be understood that some of these steps may be computer-implemented. The method 500 may include a step 510 of preparing the sample for analysis. The pathologist may prepare the patient sample and add the sample to the control device. The pathologist may add at least one immunohistochemical reporter specific to each biomarker(s) to be detected to the device. In some embodiments, the reporters may also be specific to the detection and analytical method and instrumentation used. The reporters may be any biomarker staining method. The reporters may include but are not limited to non-fluorescent stains, fluorescent stains, nanoparticles, and quantum dots. The quantum dots may include the quantum dots discussed in U.S. patent application Ser. No. 12/8764,763 filed Jul. 27, 2010, U.S. patent application Ser. No. 13/060,513, filed Sep. 39, 2009, and Emory Ref. 10156, which are incorporated by reference in their entirety.

The method 500 may further include a step 520 of imaging the control unit and patient sample. In some embodiments, the image of the control unit and patient sample may be taken in one image. In other embodiments, the image of the control unit and the image of the patient sample may be taken separately. The image may be taken by the instrumentation. In some embodiments, the image may be taken digitally.

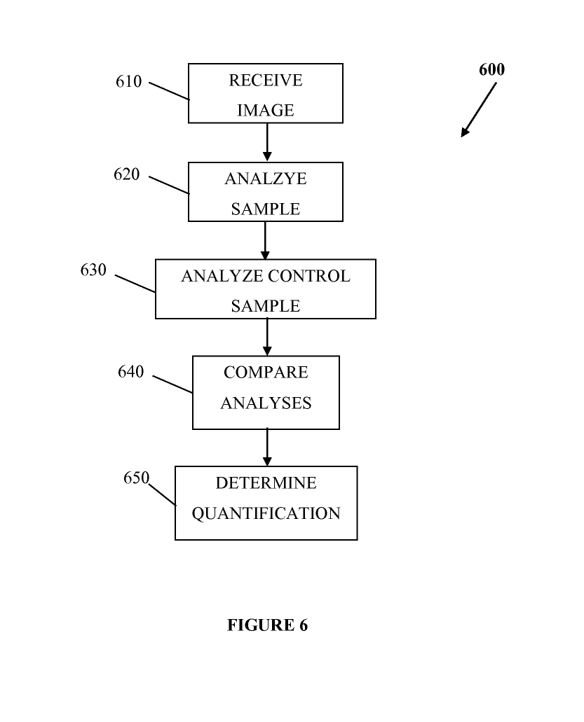

The method 500 may further include step 530 of analyzing the image. FIG. 6 shows method 600 of analyzing an image to determine biomarker concentrations according to embodiments. According to some embodiments, the analyzing method may begin after receiving the image(s) of the biological sample and control unit in step 610. The method 600 may further include the steps 620 and 630 of analyzing the biological sample and/or control unit. The steps of analyzing of the intensity the biological sample and control unit may be performed in any order and is not limited to the order of steps shown in the figure.

In some embodiments, the analyzing step 630 may include determining or measuring the intensity for each control sample. The intensities for control samples of a specific concentration may be averaged. For example, the replicates and the control sample may be averaged. Then, the intensities for the control samples may be correlated with the respective concentration to generate a calibration control. Although the calibration control is depicted as a graphical representation in the figures, the association is not limited to that graphical representation. The association may be stored and/or displayed in any form, such as a table and database.

In further embodiments, analyzing the biological sample (step 620) may include determining or measuring the intensity of at least one region of a sample. In some embodiments, specific region(s) of the sample may be analyzed. In further embodiments, the specific region is a pixel of the sample. These region(s) may be selected by the pathologist. In other embodiments, the entire sample may be analyzed.

The method 600 may further include a step 640 of comparing the analyses of the control sample(s) and the biological sample. In some embodiments, the intensity of the biological sample may be compared to the calibration control. The method may further include step 650 of generating quantification results. The generating step 650 may be based on the comparison. In some embodiments, the analyses may be compared to determine the quantitative concentration of the biological sample. In other embodiments, the intensities of a plurality of regions of the biological sample may be compared to the calibration control to generate a quantitative profile of the biological sample.

In some embodiments, the results may then be reported in step 540. In some embodiments, the information regarding the analyses (e.g., results) may be reported to the pathologist or user. The information may include information regarding the biological sample. The information may include but is not limited to the concentration of the biomarker(s) in one region of the biological sample, a profile of the concentrations of the biomarker(s) in region(s) of the biological sample, total concentration of the biomarker(s) of the biological sample, and any combination thereof. In some embodiments, the information may further include information regarding the control unit. The information may include but is not limited to the concentration and intensity profile of the control unit. The concentration may be provided in any known units, such as molarity or weight (e.g., moles or weight per volume). The concentration units may be selected by the pathologist.

In some embodiments, the information may be in a form of a report. In some embodiments, the information may be stored in a local memory. In other embodiments, the information may additionally or alternatively be transmitted to be stored in an electronic medical record for the patient, to be displayed by a display device, and/or to be printed by a printing device. In some embodiments, the information may include quantitative or quantitative profile of the biological sample. In further embodiments, the information may include the calibration control.

According to embodiments, the device including a control device can be configured to be on stained simultaneously with the patient sample and imaged and analyzed by the pathologist to calibrate staining intensity and control for variation on a slide-to-slide basis. The methods according to embodiments may analyze the staining intensity of each spot (including replicates) and generate robust standard curves with statistically significant data, taking the guess work out of expression analysis. In some embodiments, the reports may be generated based on the user or pathologist input. For example, the pathologist may select specific regions of the cell to be analyzed. Examples of the reports and analyses are shown in the bottom of FIG. 7.

The multiplexing capability enables the correlation of staining intensity to marker concentration for all markers of interest, permitting quantitative molecular profiling on a pixel-by-pixel basis in the specimen image as shown in FIG. 7. This unique design allows pathologists to perform quantitative biomarker measurements and other powerful analyses on regions of interest (or even single cells) within clinical specimens while preserving biomarker localization and structural information (see FIG. 7, right). This also allows the pathologist to view the cell at the pixel level and thus the pathologist may determine the location of the biomarker(s) with respect to each other and within the cell.

This is a significant advance over existing quantitative techniques such as ELISA assays or gene chips, which require the destruction of the patient specimen and loss of this information. These advances enable pathologists to quantitatively profile patient samples and even compare results from different staining batches or diagnostic labs.

FIG. 7 shows an example of a schematic of a pathologist workflow according the method shown in FIGS. 5 and 6. After multicolor imaging of the PTMA, the method, implemented on a computer system and stored on a computer-readable medium, may be used to correlate fluorescence intensity of each marker to concentration.

According to embodiments, the method may be used to control quality and normalize the imaged patient specimen for comparison to other samples, as well as quantitative molecular profiling while preserving marker localization and structural information (e.g. membrane staining for red channel and nuclear staining for green channel).

According to embodiments, the analyses may be compared to other analyses and concentration quantifications. In some embodiments, the analyses of the control system may be compared to a predetermined concentration panel. The predetermined concentration panel may be specific to the batch of pseudo tissue provided on the control device. In some embodiments, the batch number or identification that correspond predetermined concentration panel may be provided on the information region on the slide. The predetermined concentration panel may be stored locally on the computer or accessed remotely.

In other embodiments, the analyses of the system may be compared against each other. This may be used to validate performance of the instrumentation and reporters. For example, the performance may include that the reporter is degrading and losing its efficacy, as well as the instrumentation used (for example, the lighting devices may be degrading).

In other embodiments, the control unit may be used to validate performance of the sample preparation and/or instrumentation. In some embodiments, the control unit may establish that the proper reporter was used. For example, a positive reaction on the control unit device established that a negative result in the tissue sample is a true negative rather than being due to errors in the staining procedure.

In some embodiments, the control unit may be used as a feedback device for the computer system. In some embodiments, the computer system may use the control unit to detect and report an incorrect reporter. In other embodiments, the computer system may use the control unit to determine the operation status of the instrumentation and report the status to the user.

### Examples

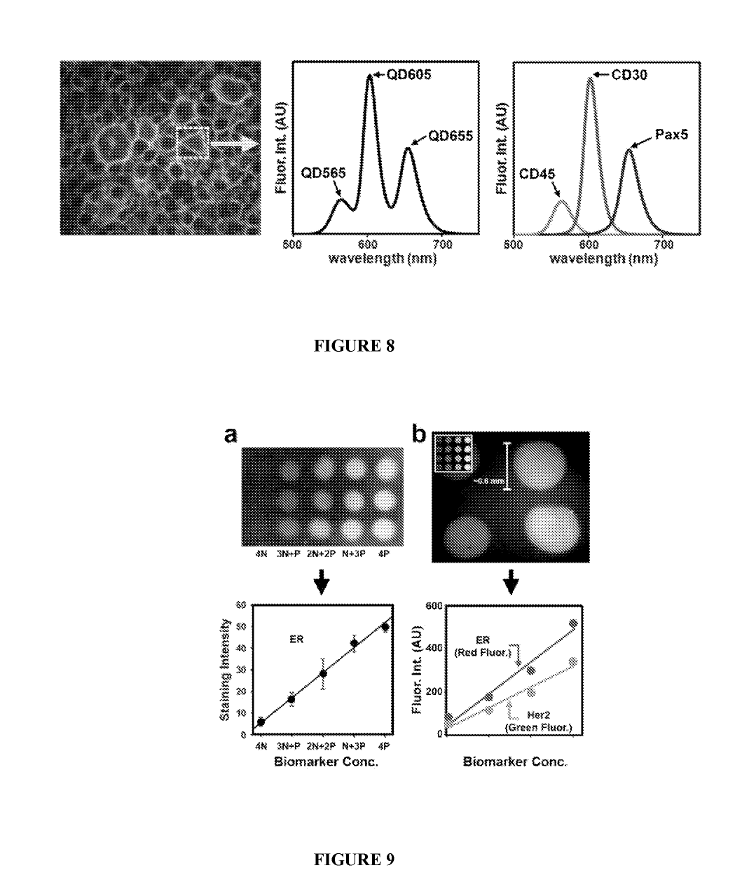

An example of analysis of a Hodgkin's lymphoma specimen using fluorescent quantum dots on a control unit according to the disclosure is shown in FIG. 8. When the control unit is used in combination with QD nanoparticle technology, quantitative molecular profiling of minute samples (FNAs) may be performed on a single slide. These QDs can have a number of advantages over existing fluorescent stains and may be simultaneously measured in patient specimens using standard fluorescence imaging or spectral hardware.

FIGS. 9A and 9B shows an example of studies for single and double marker analysis, respectively. These studies show that staining intensity in PTMA controls is highly correlated with biomarker content (R²≧0.995). The PTMA spots are prepared from ratios of low (N) and high (P) expression homogenates and evenly cover the biomarker concentration range (ex: 3N+P is a 3:1 ratio of negative to positive expression homogenates). In addition, the microarray format allows the inclusion of sample replicates to generate statistical information. This shows that quantitative multimarker analysis of PTMAs stained for two markers (using red and green QDs) is possible, with high correlation between fluorescence intensity and protein concentration for both markers. Thus, according to the embodiments of the disclosure, quantitative multimarker profiling of more than 3 markers may be possible in minute clinical specimens.

### Kits

According to some embodiments, each device may be configured to be single use. In further embodiments, each device may be individually sterilized.

In some embodiments, the devices may be sold in a kit. In some embodiments, the kit may include at least one device. In some embodiments, the kit may include more than one device.

In further embodiments, the kit may include a plurality of devices. Each device may include a control region and a tissue specimen region, according to embodiments, for example, as shown in FIG. 2.

In some embodiments, the kits may be specific to the biomarker(s) to be detected. In further embodiments, the kits may be further specific to the reporter(s) to be used.

In some embodiments, the kit may further include at least one reporter to be used with the devices.

While the disclosure has been described in detail with reference to exemplary embodiments, those skilled in the art will appreciate that various modifications and substitutions can be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. A device configured for immunohistochemical tests of a biological sample using at least one reporter specific to at least one biomarker, comprising:
a substrate, the substrate including a control region and a biological sample region, the control region including a control unit, the control unit including a plurality of control samples of pseudo-tissue,
each control sample including at least one of a first material and a second material, the first material including one or more homogenates that are negative for the at least one biomarker, and the second material including one or more homogenates that are positive for the at least one biomarker.

2. The device according to claim 2, wherein each sample is of different concentrations of the first and second materials.

3. The device according to claim 2, wherein the samples are disposed on the control unit in order of highest negative concentration to lowest positive concentration.

4. The device according to claim 2, wherein the control unit further includes at least one replicate sample for each control sample.

5. The device according to claim 4, wherein the samples and replicate samples are disposed in a microarray.

6. The device according to claim 5, wherein the samples are disposed on the control unit in order of highest negative concentration to lowest positive concentration in a first direction.

7. The device according to claim 6, wherein each replicate sample is disposed on the control unit in a second direction adjacent to respective control sample.

8. The device according to claim 1, wherein each sample of the control unit is substantially homogenous.

9. The device according to claim 1, wherein each sample of the control unit is configured to have substantially uniform intensity throughout the sample.

10. The device according to claim 1, wherein the control unit include at least three different control samples.

11. The device according to claim 1, wherein control samples are configured to test at least two different biomarkers.

12. A kit for immunohistochemical tests of a biological sample using at least one reporter, comprising:
a plurality of control devices,
each device including: a substrate, the substrate including a control region and a biological sample region,
the control region including a control unit, the control unit including a plurality of control samples of pseudo-tissue, and
each control sample including at least one of a first material and a second material, the first material including one or more homogenates that are negative for the at least one biomarker to be tested, and the second material including one or more homogenates that are positive for the at least one biomarker to be tested.

13. The kit according to claim 12, further comprising:
the at least one reporter, wherein the at least one reporter is quantum dots.

14. The kit according to claim 12, wherein each sample is configured to have substantially uniform intensity.

15. A method for determining concentration of at least one biomarker in a biological sample on a control device comprising:
receiving an image of the control device prepared with at least one reporter specific to each at least one biomarker, the image including an image of the biological sample and of a control unit, the control unit including a plurality of control samples of a pseudo-tissue of different concentrations of the at least one biomarker, and at least one replicate sample for each control sample;
analyzing the image of the control unit, the analyzing the image of the control unit include determining the intensities of each control sample and corresponding replicate sample and averaging the intensities of control and replicate samples for each concentration;
correlating the averaged intensities to each concentration;
analyzing the image of the biological sample to determine intensity of at least one region;
comparing the intensity of the at least one region to the correlated intensities and concentrations of the control unit; and
determining the concentration of the biomarker in the at least one region;
reporting the concentration of the biomarker.

16. The method according to claim 15, wherein each sample of the control unit has substantially uniform intensity.

17. The method according to claim 16, wherein more than one biomarker is analyzed simultaneously.

18. The method according to claim 15, wherein:
the at least one reporter is a fluorescent stain specific to each biomarker; and
the correlating includes simultaneously correlating the averaged intensities for each biomarker to each concentration.

19. The method according to claim 15, wherein the reporter is quantum dots.

20. The method according to claim 15, wherein the reporting includes generating a concentration profile of the biological sample.
